(19) United States
(12) Patent Application Publication
(10) Pub. No.: US 2003/0236506 A1
(43) Pub. Date: Dec. 25, 2003

(54) Dual outside diameter cannula for insertion into bone

(76) Inventors: Eric Schofield, Richmond, VA (US); Roy Lim, Cordova, TN (US); Michael C. Sherman, Memphis, TN (US)

Correspondence Address: COATS & BENNETT, PLLC, P O BOX 5, RALEIGH, NC, 27602, US

(21) Appl. No.: 10/176,223

(22) Filed: Jun. 20, 2002

(51) Int. Cl. A61M 5/32

(52) U.S. Cl. 604/272; 606/167; 604/164.01; 264/632

(57) ABSTRACT

A cannula and a method of inserting the cannula into a bone within a patient. The cannula comprises a distal section, a proximal section, and an intermediate section positioned therebetween. The distal section has a smaller diameter than the proximal section. The smaller diameter assists in inserting the distal section into the bone. The increased diameter of the proximal section gives rigidity and strength to prevent bending or flexing during insertion. The proximal section has a wall thickness greater than the distal section to give rigidity and strength to the cannula.

### BACKGROUND OF THE INVENTION

Various medical procedures require a physician to obtain a sample of a patient's bone or penetrate to the bone marrow cavity to extract bone, bone marrow or bone marrow cavity fluids. Examples include diagnostic tests and determining the suitability of the patient as a transplant donor.

The procedures require the physician to use a sharpened instrument to penetrate the hard, outer layer of the bone. One type of sharpened instrument includes a stylet fitted within a cannula. The procedures require the instrument to have a combination of attributes including rigidity to prevent bending and breaking while being inserted into the bone, and be of a minimum size to prevent unnecessary damage to the bone and surrounding tissue.

Prior art instruments have been designed to be constructed of a flexible material to be inserted within soft tissue, veins, and arteries to access specific areas within the patient. These devices are not applicable to penetrating bone because the flexible construction does not have the necessary rigidity to penetrate through the hard outer layer of the bone.

Other biopsy needles are constructed of a rigid material for penetrating into the bone. These needles have a tapered tip to facilitate insertion into the bone and a constant outer diameter extending along the length. The outer diameter is sized such that the device has adequate rigidity and strength to be inserted into the bone without bending or flexing. However, the enlarged size may result in unnecessary damage to the bone and the surrounding tissue.

### SUMMARY

The present invention is directed to a cannula for insertion into bone. In one embodiment, the cannula includes a distal section having a first outer diameter and a proximal section having an enlarged outer diameter. The sizing is important because the reduced size of the distal section allows for penetrating into the bone without causing unnecessary damage. The enlarged proximal section allows for support to prevent bending when the distal end is inserted into the bone.

In another or the same embodiment, the distal section has a first wall thickness. The proximal section has a larger wall thickness to further prevent the cannula from bending when in use.

An intermediate section may be positioned between the distal section and the proximal section. The intermediate section may have a tapered configuration such that the outer diameter tapers from the size of the proximal outer diameter to the size of the distal outer diameter. In another embodiment, a tip is positioned on the end of the distal section to facilitate penetration into the bone. The tip may be tapered, and may include a sharpened edge.

In use, the cannula is handled such that the distal end penetrates into the bone. The sections of the cannula having the larger diameter do not penetrate the bone. The distal section includes a length with a constant diameter. Increased penetration into the bone results in a longer opening, without an increase in the diameter of the opening within the bone.

### BRIEF DESCRIPTION OF THE DRAWINGS

FIG. 1 is a perspective view illustrating one embodiment of a cannula constructed in accordance with the present invention;

FIG. 2 is a partial perspective view of one embodiment of a cannula with a stylet extending outward from the distal end in accordance with the present invention;

FIG. 3 is a cross-sectional view of the cannula of FIG. 1 cut along line 3-3; and

FIG. 4 is a cross-sectional view of the distal section of FIG. 3 cut along line 4-4;

FIG. 5 is a cross-sectional view of the proximal section of FIG. 3 cut along line 5-5;

FIG. 6 is a cross-sectional view of an alternative embodiment of the present invention illustrating the cannula constructed of different materials;

FIG. 7 is a side view illustrating the cannula nearing insertion into the patient in accordance with one embodiment of the present invention;

FIG. 8 is a side view illustrating the cannula nearing insertion into the bone in accordance with one embodiment of the present invention; and

FIG. 9 is a side view illustrating the distal section of the cannula inserted within the bone with the intermediate section and the proximal section to the exterior of the bone in accordance with one embodiment of the present invention.

### DETAILED DESCRIPTION

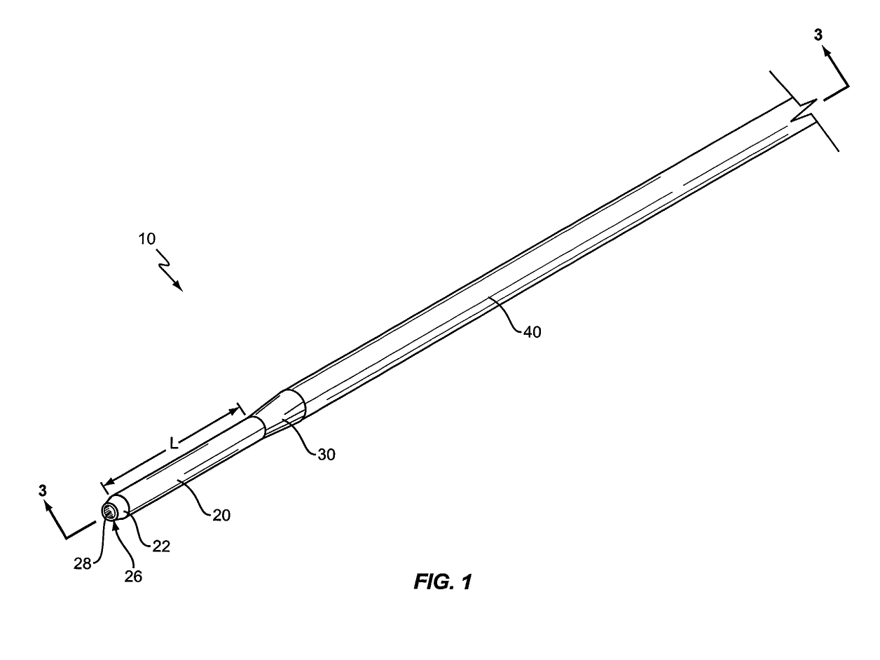

The present invention is directed to a cannula, generally illustrated 10 in FIG. 1, and a method of inserting the cannula into a bone within a patient. Cannula 10 comprises a distal section 20, a proximal section 40, and an intermediate section 30 positioned there between. The distal section 20 has a smaller outer diameter than the proximal section 40. The smaller outer diameter assists in inserting the distal section 20 into the bone. The proximal section 40 has a larger wall thickness than the distal section 20 to give rigidity and strength to prevent bending or flexing during insertion.

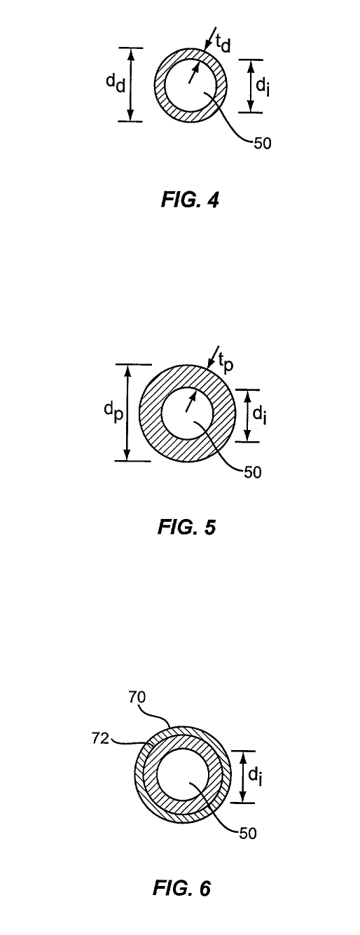

The distal section 20 includes a distal end 26 having an opening 28 through which a stylet 60 extends. The distal section 20 has a length L which may have a variety of sizes depending upon the application. In one embodiment, the length L of the distal section 20 is about 1.0 inch. The outer diameter d_{d} (FIG. 4) over the length L is substantially constant. In one embodiment, the distal end is 11 gauge and has an outer diameter d_{d} of about 0.120 inches. The wall thickness t_{d} of the distal section 20 is illustrated in FIG. 4. Wall thickness t_{d} may vary depending upon the application. In one embodiment, wall thickness t_{d} is about 0.027 inches. The wall thickness t_{d} may be constant over the length L, or may vary. In one embodiment illustrated in FIG. 3, wall thickness t_{d} is substantially constant over the length L. In another embodiment (not illustrated), wall thickness t_{d} gradually increases over the length L with the smallest thickness adjacent the distal end 26 and the largest thickness adjacent the intermediate section 30.

An inwardly tapered tip 22 may be positioned at the end of the distal section 20 adjacent to the opening 28. Tapered tip 22 may include a sharpened edge to facilitate insertion of the cannula 10 into the patient.

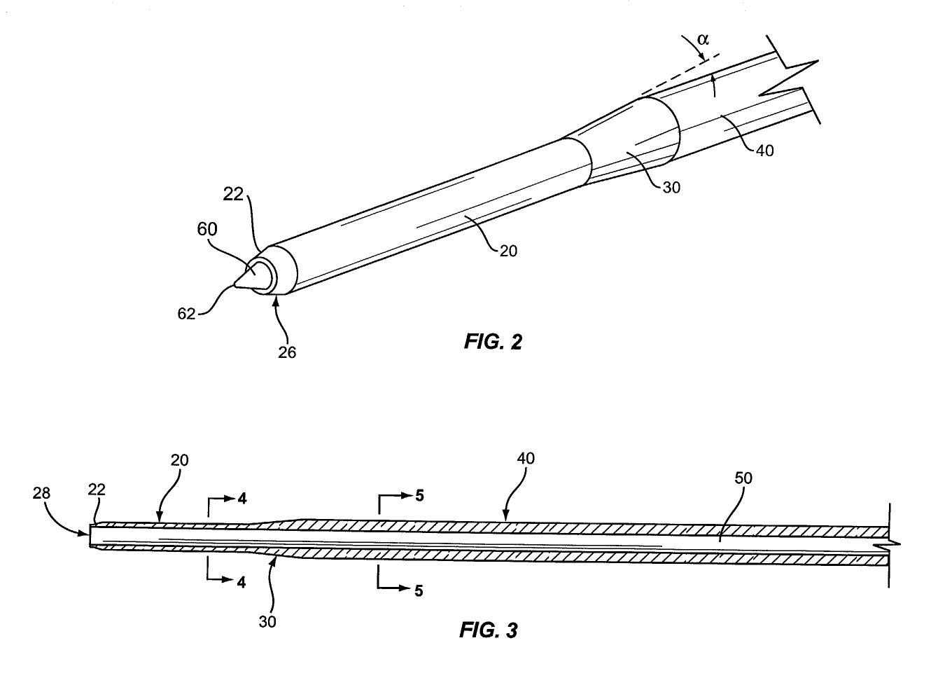

Intermediate section 30 is positioned between the distal section 20 and proximal section 40. Intermediate section 30 has a tapering outer diameter that ranges in size between the outer diameter of the proximal section 40 to the outer diameter of the distal section 20. In one embodiment, the intermediate section tapers from an outer diameter of about 0.165 inches to about 0.120 inches. The amount of taper and length may vary depending upon the application. In one embodiment as illustrated in FIG. 2, the taper angle α is about 10°. The wall thickness of the intermediate section varies across the length in a gradual manner from the smallest wall thickness adjacent to the distal section 20 and the largest adjacent to the proximal section 40.

In one embodiment, the proximal section 40 has a larger wall thickness than the distal section 20. The additional thickness increases the rigidity of the proximal section 40 to reduce flexing and bending during insertion of the cannula 10 into the bone. The wall thickness tₚ of the proximal section 40 may be within a wide range depending upon the application. In one embodiment, the wall thickness tₚ is about 0.072 inches. The wall thickness tₚ may be constant over the length of the proximal section 40 as illustrated in FIG. 3. In another embodiment, the wall thickness tₚ may vary along the length. In one embodiment, the wall thickness is constant over the distal, intermediate, and proximal sections.

Proximal section 40 has a larger outer diameter dₚ (FIG. 5) than the outer diameter d_{d} of the distal section 20. In one embodiment, the outer diameter dₚ is about 0.165 inches. The outer diameter dₚ may be constant over the length of the proximal section 40 as illustrated in FIG. 3. In another embodiment, the outer diameter dₚ varies over the length.

The cannula 10 includes a lumen 50 extending the length. The lumen 50 is sized to receive a stylet 60 that extends the length of the cannula 10 and through the opening 28 in the distal end 26. In one embodiment as illustrated in FIG. 4, an inner diameter dᵢ of the lumen 50 is substantially constant the entire length of the cannula 10. In one specific embodiment, the inner diameter is about 0.093 throughout the length of the cannula 10. In another embodiment, the inner diameter dᵢ may vary over the length. The inner diameter dᵢ may have a variety of sizes depending upon the application.

Cannula 10 may be constructed in a number of different manners. In one embodiment, cannula 10 is constructed from a single piece of material, such as stainless steel. The cannula 10 may further be constructed of any metal that offers rigidity for inserting the cannula 10 into the bone. In one embodiment, cannula 10 is constructed of titanium to be compatible with MRI equipment. In an alternative embodiment as illustrated in FIG. 6, cannula 10 is constructed of outer and inner materials 70, 72. In one embodiment, the outer material 70 forms an outer shell around the inner material 72. The outer material 70 has a rigid construction to prevent bending or flexing of the cannula 10 during insertion into the bone. Inner material 72 may further be constructed to add rigidity.

A stylet 60 may be inserted within the lumen 50 as illustrated in FIG. 2. The elongated stylet 60 extends the length of and is slideably received within the lumen 50. The stylet 60 extends through the opening 28 at the distal end 26 and provides a smooth external profile between the cannula 10 and stylet 60 to facilitate penetration into the bone. Stylet 60 includes a cutting edge 62 at the distal end. Cutting edge 62 that may have a variety of orientations and dimensions to facilitate bone penetration.

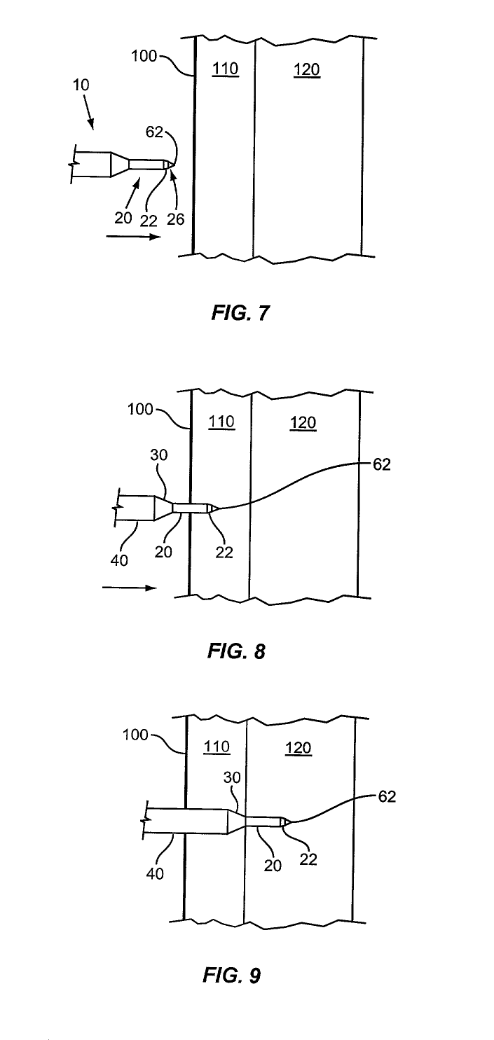

FIGS. 7, 8, and 9 illustrate the use of the cannula 10. FIG. 7 illustrates the cannula 10 positioned adjacent to the patients skin 100, tissue 110, and bone 120. In this embodiment, the cannula 10 is inserted through the skin 100 and tissue 110. In other embodiments, the skin 100 and tissue 110 may be resected prior to the use of the cannula 10 such that only the bone 120 is contacted. Stylet 60 is inserted within the cannula 10 with the cutting edge 62 protruding through the opening 28 for facilitating insertion.

FIG. 8 illustrates a stage during the insertion process. The distal section 20 has penetrated through the skin 100 and into the tissue 110. The intermediate section 30 and proximal section 40 have yet to enter into skin 100. FIG. 9 illustrates the cannula 10 with stylet with cutting edge 62 inserted into the bone 120. The cannula 10 has been inserted a distance into the patient such that the distal section 20 is the only portion of the cannula 10 penetrating into the bone 120. Neither the intermediate portion 30 nor proximal section 40 penetrate the bone 120. The intermediate portion 30 and proximal section 40 penetrate through the skin 100 and into the tissue 110. The smaller outer diameter d_{d} of the distal section 20 prevents unnecessary damage to the bone that could occur if the intermediate section 30 or proximal section 40 were inserted. The increased wall thickness tₚ of the proximal section 40 prevents the cannula 10 from bending such that the force applied to the cannula 10 is directed to penetration into the bone 120.

In the embodiment illustrated in FIGS. 7, 8, and 9, stylet 60 also penetrates into the bone 120 as it extends from the opening 28 in the distal end 26. In another embodiment, there is no stylet 60 and only the cannula 10 is inserted into the bone 120.

The cross-section shape of the distal 20, intermediate 30, and proximal 40 sections may have a variety of different configurations. In one embodiment, each section is substantially circular. In one embodiment, the sections are rectangular. In another embodiment, sections are oval. In another embodiment, sections are triangular. The different sections may have different cross-sectional shapes. In one embodiment, distal 20 and proximal 40 sections have a first cross-sectional shape, and the intermediate section 30 has a second, different cross-sectional shape. The term “diameter” is used herein to mean the size of the device by a straight line passing through a center of the cross-sectional shape. The term “diameter” is used to include circles, as well as other shapes.

One embodiment of a cannula 10 includes a distal section 20 having a length of about 1.0 inches, an outer diameter of about 0.120 inches, and an inner diameter of about 0.093 inches. The proximal section 40 has an outer diameter of about 0.165 inches, an inner diameter of about 0.093 inches. The intermediate section has a tapered outer diameter that ranges from a first edge of about 0.165 inches to a second edge of about 0.120 inches. The intermediate section 30 tapers at about a 10° angle relative to the proximal section 40. A constant inner diameter lumen 50 of about 0.093 inches extend the entire length of the cannula. The distal section 20, intermediate section 30, and proximal section 40 have a combined length of about 5.0 inches.

The present invention may be carried out in other specific ways than those herein set forth without departing from the scope and essential characteristics of the invention. Proximal section 40 may have a variety of lengths depending upon the application. A handle or other type of holding device may be mounted to the proximal section 40 for handling by the physician. The handles are well known in the art and are not considered part of this invention. The present embodiments are, therefore, to be considered in all respects as illustrative and not restrictive, and all changes coming within the meaning and equivalency range of the appended claims are intended to be embraced therein.

What is claimed is:

1. A cannula for inserting into bone comprising:
an elongated distal section having a constant distal outer diameter;
a proximal section having a proximal outer diameter, the proximal outer diameter being larger than the distal outer diameter; and
an intermediate section positioned between the distal section and the proximal section, the intermediate section having a variable outer diameter that ranges from the distal outer diameter to the proximal outer diameter;
the distal section, the proximal section, and the intermediate section having a rigidity to be inserted into the bone.

2. The cannula of claim 1, wherein a wall thickness of the proximal section is greater than a wall thickness of the distal section.

3. The cannula of claim 1, wherein the proximal outer diameter is constant.

4. The cannula of claim 1, further comprising a constant inner diameter extending through the distal section, the intermediate section, and the proximal section.

5. The cannula of claim 1, further comprising a variable inner diameter extending through the distal section, the intermediate section, and the proximal section.

6. The cannula of claim 1, wherein the distal section has a length of about 1 inch.

7. The cannula of claim 1, wherein the distal outer diameter is about 0.120 inches.

8. The cannula of claim 1, wherein the proximal outer diameter is about 0.165 inches.

9. The cannula of claim 1, wherein the distal outer diameter is about 0.120 inches.

10. The cannula of claim 1, further comprising a tapered tip positioned at an end of the distal section.

11. The cannula of claim 1, wherein the proximal outer diameter is about 40% greater than the distal outer diameter.

12. The cannula of claim 1, wherein the intermediate outer diameter tapers at an angle of about 10% relative to the proximal section.

13. The cannula of claim 1, wherein a wall thickness is constant through the proximal section, intermediate section, and distal section.

14. A device for inserting into bone comprising:
a distal section having a first outer diameter and a first wall thickness;
a proximal section having a second wall thickness which is greater than the first wall thickness, the proximate section having an outer diameter greater than the first outer diameter; and
the distal section and the proximal section being constructed of a rigid material to prevent bending when the distal section is inserted into the bone.

15. The device of claim 14, further comprising an intermediate section positioned between the distal section and the proximal section, the intermediate section having an inwardly tapering intermediate wall thickness and a tapering intermediate wall thickness.

16. A cannula comprising:
a distal section comprising a constant distal outer diameter and a constant distal wall thickness;
an intermediate section comprising a tapering intermediate outer diameter and a tapering intermediate wall thickness; and
a proximal section comprising a proximal outer diameter greater than the distal outer diameter, the proximal section comprising a proximal wall thickness greater than the distal wall thickness.

17. A method of inserting a cannula into a bone comprising the steps of:
inserting a distal end of a cannula into a surface of a bone; and
inserting a distal section of the cannula beyond the surface of the bone, the distal section extending from a first point to a second point with a substantially constant diameter therebetween.

18. The method of claim 17, wherein the step of inserting a distal end of the cannula into the surface of the bone comprises inserting a tapered end of the cannula into the bone.

19. The method of claim 17, further comprising maintaining a proximal section of the cannula on an exterior of the bone, the proximal section having a larger outer diameter than the distal end.

20. The method of claim 17, further comprising reinforcing a proximal section of the cannula to prevent bending and flexing of the cannula.

21. The method of claim 17, wherein the second point of the cannula is an intermediate section having a larger diameter than the distal section.

22. A method of supporting a stylet for penetrating a bone comprising the steps of:
encasing a distal section of a stylet with a first wall thickness;
encasing a proximal section of the stylet with a second thickness which is greater than the first thickness; and
inserting the stylet into a patient with the distal section penetrating a bone and the proximal section positioned out of the bone.

23. The method of claim 22, wherein the step of encasing a distal section of the stylet with the first wall thickness comprises positioning a tip of the stylet unobstructed.

24. A method of forming a cannula for inserting into a bone comprising the steps of:
forming a hollow tube with a first end having a first outer diameter;
forming the hollow tube to have a second end having a second outer diameter that is greater than the first outer diameter;
forming the hollow tube to include the first end having a smaller wall thickness than the second end; and
forming the hollow tube from a material that will not bend when the first end is inserted into bone.
